(19) United States
(12) Patent Application Publication
(10) Pub. No.: US 2023/0190334 A1
(43) Pub. Date: Jun. 22, 2023

(54) TWO-STAGE PRECISION GUIDE AND METHOD FOR FIXING TO A LOW-HEIGHT MAXILLARY BONE TRIPOD WITH SHORT DENTAL IMPLANT FUNCTION

(71) Applicant: OSCAR RUESGA DELGADO, MADRID (ES)

(72) Inventor: OSCAR RUESGA DELGADO, MADRID (ES)

(21) Appl. No.: 16/479,566

(22) PCT Filed: Jan. 17, 2018
(86) PCT No.: PCT/ES2018/070033

§ 371 Date: Jul. 19, 2019

(30) Foreign Application Priority Data
Jan. 19, 2017 (ES) P201730060

(51) Int. Cl. A61B 17/68; A61B 17/17

(52) U.S. Cl. CPC A61B 17/68; A61B 17/17; A61B 17/8033

(57) ABSTRACT

A two-stage precision guide and a method for fixing to a tripod with short dental implant function. The two-stage precision guide includes a fixation guide and a drilling guide. In a first stage of use, the fixation guide is adhered to the tripod allowing the body of the tripod, containing five perforations and having three identical feet, to be positioned on the bone. In the second stage of use, once the body has been fixed to the bone, the two-stage guide allows the bone to be worked on using the drilling guide. Once the holes have been made, the two parts of the two-stage guide are removed to be subsequently fixed to the bone by the body. With the feet being placed in the bone, a dental prosthesis can be situated on the tripod thereby allowing the tripod to act as a short dental implant.

### OBJECT OF THE INVENTION

10 The present invention belongs to the dental implantology sector.

The object of the present invention is a two-stage precision guide and method for fixing to a low-height maxillary bone a tripod with short dental implant function which provides a convenient, quick and safe way to fix a tripod, that can support the load of a prosthesis, in areas of the jaw bone wide but low, avoiding regenerative surgeries such as sinus lift or block maxillary reconstructions.

### BACKGROUND OF THE INVENTION

20 Short dental implants are implants of relatively recent use. These are wide dental implants, usually greater than 5MM's width and short, usually up to 5MM. These implants are aimed at short and wide areas of bone but, although presenting an acceptable osseointegration, they are very unpredictable when putting a prosthetic load on them, which significantly reduces its use. The Roxolid® SLActive® implant with short Loxim™ 25 from Straumann and the Anyridge® short implants from IDS are an example of existing short implants. The microimplants are very thin implants of between 1.3MM to 2MM in width and lengths of approximately 10MM whose use is exclusive in orthodontic treatments since they only support tensile forces of up to 300g. The Unitek™ implant from 3M is an example of an existing microimplant.

There are still many inventions that seek to combine a central dental implant with one or more finer implants that go through it, inventions that seek to provide more stability to the first implant, although none of them provides a concrete, simple, practical solution, feasible application and clinical manipulation of the device, being many of them 35 impossible to perform and impossible to put on the lips or simply unrealistic. In addition, many of them do not provide a universal prosthodontics solution because they carry in their design a pillar for cemented or prosthetic anchoring. Some of these patents I quote them below:
The U.S. Pat. No. 2,857,670A by the author TF Kiernan, Jr with date of publication of Oct. 28, 1958 and U.S. Pat. No. 5,890,902A by the author SAPIAN SL with date of publication of Apr. 6, 1999 both similar, where the authors seek to provide stability to a dental implant by pivots which are perpendicular to the axis of the implant. Besides this implant, is very difficult to perform and unfeasible from a clinical point of view since it would considerably damage the bone and it would be very difficult to remove it if there is any problem.
The U.S. Pat. No. 3,579,831A by the authors IRVING J STEVENS and JERRY ALEXANDER with publication date of May 25, 1971. Here the authors seek to stabilize a dental implant by means of two thin transverse sticks, which lacks clinical and practical sense.
The Patents: U.S. Pat. No. 3,981,079 (A) of the author LENCZYCKI J J with publication date of Sep. 21, 1976; FR2720264A1 of the authors BOMPARD and MAUDUECH E with date of publication of Dec. 1, 1995; U.S. Pat. No. 5,542,847A from the authors KADAR A and MARGULIES J Y with publication date of Aug. 6, 1996; ATA1212000 by the authors HAAS ROBERT DR and WATZEK GEORG DR with publication date of Sep. 15, 2000; FR2797171A1 by the authors BOMPARD BRUNO JACQUES HUBERT and BOMPARD ELISABETH CATHERINE MA with publication date of 9 Feb. 25, 2001; US2002031747A1, EP1330207A2 of the company ADVANCED DENTAL ENG LTD with publication date of Mar. 14, 2002; FR2848096A1 by author HOFMANN B with publication date of Jun. 11, 2004; FR3006883 (A1) by the authors DADAKARIM and LOUIS JOEL with a submission date of Dec. 19, 2014, all of these patents seek to block and fix an implant by crossing it perpendicularly with a microimplant and providing many of them with technical and clinical use solutions. All these devices are clinically unviable but interesting, although they do not correspond with the patent claimed here.
The patent KR20110000660A of the author SEVRAIN LIONEL C with publication date of Jan. 4, 2011 tries to provide a practical solution for clinical application, although this solution is very complex and clinically unfeasible since it intends to introduce microimplants in the biological space and the cortical bone area that should not be invaded by a dental implant.
The patent US2015134016A1 of the company BIEDERMANN TECHNOLOGIES GMBH & CO with publication date of May 14, 2015 that has a purpose other than the one claimed here but seeks to apply a tripod in the bone by inserting sticks around a central pivot to leave them in the bone and anchoring it after bone regeneration surgeries, all this through a practical guide. The guide of this design does not have the indicated precision for the implantology, neither the connection is applicable, besides inserting some micropins or toothpicks that are useless to support prosthodontic loads.
The patent ES2160498 (A1) of the author HERNANDEZ CABRERA J M with date of publication of Nov. 1, 2001 patent that although ingenious is unworkable from a manufacturing and clinical application point of view.
The patent WO2009044395 of MEDILOCK MEDICAL SOLUTIONS LTD company with date of publication of Apr. 9, 2009 that seeks to block in the bone a bolt through another, with a very generic description and without any practical solution, this patent is also not focused to the field of oral implantology.
The U.S. Pat. No. 5,564,925 of the author SHAMPANIER A with date of publication of Oct. 15, 1996, although very creative, interesting and with the same purpose as the patent claimed here, this design is technically unviable in all respects since it does not provide the adequate stability for support the prosthodontic occlusal load, does not provide an application technical solution and does not have a universal or viable prosthetic solution.
The U.S. Pat. No. 3,955,280 of the author SNEER M with date of publication of May 11, 1976 patent very original but technically unfeasible with a unique prosthetic solution and not universal
The patent EP3075346 of the author KOSTAKIS G with publication date of Jun. 5, 2016 provides a practical and interesting solution but technically unfeasible since the insertion of the lateral microimplants is done in the biological space and the cortical bone, space that should not be play under no circumstances.
The U.S. Pat. No. 5,984,681 of the author HUANG B K with publication date of Nov. 16, 1999 presents in broad strokes the insertion of micropins in an external connection implant without providing clinical solutions or methodology besides being technically unfeasible in the presentation made.
The patent CN101947139 by the same author and published on Jan. 19, 2011 presents a simplified but very unstable implant model with a unique, non-universal connection.
My patent ES1153433 with a filing date of Mar. 10, 2016 and my utility model U201631447 with a filing date of Dec. 18, 2016, are an incomplete and imprecise variant of the patent claimed here, patent that perfects in all the senses the technical realization of the device and simplifies to the maximum its clinical use, presenting some improvements that make it unique as we see below.

### DESCRIPTION OF THE INVENTION

To correct the above-described defects, the invention proposes a simple invention to make and use, realistic, very versatile, ergonomic, respecting established technical standards and very original. The invention is a two-stage precision guide and method for fixing to a low-height maxillary bone a tripod with short dental implant function which is composed of two inseparable and indispensable elements for the realization and comfortable use of it: the fixing guide and the drill guide. These elements must be designed with a geometry and precise measurements for the proper functioning of the device. This two-stage precision guide allows to fix a tripod constituted by a central body and three legs and, as its name indicates, the guide consists of two phases of use.

In the first phase of use of the two-stage precision guide or fixation phase of the tripod's body, we use the fixation guide and the tripod's body, joined the first to the second by a simple cylindrical head's screw that has a narrower cylindrical stem with external helical threads. This screw also has a polyhedral cavity in its head that serves to manipulate it with the appropriate instrument. The fixation guide, whose function is to accurately transport and fix the tripod's body in the low-lying maxillary bone, is a solid block-shaped cup without stem whose foot is polyhedral and whose calyx is cylindrical containing an opened polyhedral cavity. The foot of the guide has five holes. Three of these holes are inclined, they are cylindrical, identical between them and their central axes cross with the central axis of the guide at the same point above the object. These holes do not touch each other and cross the foot completely. The entrance's centres of these holes, located in the base superior of the foot of the guide, draw an equilateral triangle and, the exit's centres of these holes, located in the inferior base at the foot of the guide, draw an equilateral triangle larger than the one described above. Between two of these holes and without touching them, there is a fourth cylindrical hole that's parallel to the central axis of the guide. This fourth hole is narrowed inside by an annular edge perpendicular to the central axis of this hole, this turns it into a new cylindrical hole of smaller diameter and so, the entrance of the fourth hole located in the upper base of the foot of the guide is wider than the exit of it, located in the lower base of the foot of the guide. These four holes surround without touching it a fifth polyhedral hole, perforated from the centre of the upper base of the foot's guide downwards. This hole doesn't cross the foot completely. Above and around this foot is the chalice of the guide which is a cylindrical ring that contains a polyhedral cavity open upwards and delimited from below by the upper base of the foot's guide. They are three rails attached to the outside of this ring that lead the user's guide, as explained later in this description. These are parallel to the central axis of the fixation guide and are each located in one of the three sagittal planes of the three inclined holes mentioned above.

This guide covers the body of the tripod and is fixed by the cylindrical and simple head screw. This body is a solid, cylindrical object with the shape of a wide-floor glass.

This glass contains a polyhedral cavity open upwards, surrounded by a cylindrical wall and superimposed on the floor of the cylindrical floor of the glass. This cavity is the antagonist of the fixation guide's foot. On the outside this wall is smooth and this floor is rough and has external helical threads. Inside this floor is perforated by five perforations that start from the bottom of the glass's cavity, bottom that is also the upper base of the glass's floor. Three of these perforations are inclined, cylindrical, identical between them and their central axes intersect with the central axis of the glass at the same point above the object. These perforations do not touch each other. The entrance's centres of these perforations, located in the bottom of the polyhedral cavity of the glass, draw an equilateral triangle. The outlets of these perforations are located on the sides or at the bottom base of the floor of the glass and their centres also draw an equilateral but larger triangle than the aforementioned one.

These three perforations can be composed of two cylindrical spaces, the upper one wider than the lower, separated by an annular edge perpendicular to the central axis of said perforations. Alternatively, the narrower lower space may contain propeller threads. These three perforations can also be constituted of a single cylindrical space with smooth walls that contain ditches that start from their entrances, that do not touch each other, that are parallel to the central axes of the perforations, that do not cover them in their entirety and which serve to attach the rails of the legs of the tripod on them as we will see later in this description. These two configurations of the three inclined perforations of the tripod's body define two types of bodies, which differ only by this feature. The entrances of these three perforations of the glass have the same width as the exit of the three inclined holes of the fixing guide and serve them as a continuation downwards when the two elements are adhered. This is possible since the entrance and exit's centres of the fixation guide's inclined holes as well as the entrance and exit's centres of the tripod's body inclined perforations draw an overlap of equilateral triangles of increasing size, from top to bottom, when the fixation guide and the glass are tightly joined.

Between two of these glass's inclined perforations and without touching them, there is a fourth cylindrical perforation, parallel to the central axis of the glass and which does not cross its floor completely. This fourth perforation of the glass has the same width as the outlet of the fourth perforation of the fixing guide and serves as a continuation downwards when this guide is inserted in the glass. This fourth perforation of the floor of the glass has a wall containing internal screw threads where the above-mentioned single head screw is screwed. These four perforations surround without touching it a fifth cylindrical perforation, located in the centre of the glass's floor, perforation that does not cross the floor and that is surrounded by a wall with internal helix threads. This is used to attach the correct dental prosthesis to the body of the tripod, using a single head screw. In this first phase of use of the two-stage guide both the fixation guide and the body of the tripod are perfectly joined, the guide's foot inserted inside the glass's cavity and the calyx's lower base of the fixation guide resting on top of the guide.

The arrangement of the three guide rails mentioned above, allows the user to intuit where the tripod's body will be positioned. The user will insert it precisely into the bone, avoiding anatomical areas of risk, either visually or by surgical splint. In the second phase or phase of insertion of the legs of the tripod, once the tripod's body is placed in the bone, by means of the appropriate instrument, the screw that fix the fixing guide to the tripod's body is removed. With the fixing guide still in the tripod's body, we fix the drill guide to the fixing guide end the tripod's body with a long and stepped head screw.

The drill guide is a polyhedral slab with the exact polyhedral shape of the fixing guide's polyhedral cavity where it will be placed. This slab has a polyhedral pivot protruding from the centre of its bottom base downwards. This pivot will correspond to the fifth polyhedral hole of the fixing guide and serves to place the drill guide with precision and stability inside the fixing guide. Above and in a corner of the upper base of the slab is another protruding pivot or handle with polymorphous shape that serves to grip and transport the drill guide. This drill guide contains three inclined, cylindrical and identical holes. Its central axes intersect with the central axis of the drill guide at the same point above this object. These holes do not touch each other and cross the drill guide completely. The centres of the inlets of these holes, located in the upper base of the drill guide, draw an equilateral triangle and, the centres of the outlets of these holes, located in the lower base of the drill guide, draw a larger equilateral triangle. The width of these holes is less than the width of the inclined holes of the fixing guide and of the tripod's body inclined perforations. The outlets of these drill guide's three inclined holes serve as a continuation upwards of the fixing guide's three inclined holes entrances and therefore also of the tripod's body three inclined holes entrances when the three elements are attached. This is possible since the entrances and exits centres of the drill guide's inclined holes, the entrances and exits centres of the fixing guide's inclined holes and the entrances and exits centres of the tripod's body inclined perforations draw a superposition of equilateral triangles of increasing size, from top to bottom, when the drill guide, the fixing guide and the tripod's body are closely joined.

Between two of the drill guide's inclined holes and without touching them, there is a fourth cylindrical hole, parallel to the central axis of the drill guide that cross it completely. This hole is narrowed inside by an annular edge that's perpendicular to the central axis of the hole reducing it to a smaller diameter's hole and so the entrance of the hole, located in the upper base of the drill guide is wider than the hole's exit located at the bottom base of this guide. This outlet also has the same width as the entrance of the fixing guide's fourth hole. To fix the drill guide and the fixing guide perfectly together with the tripod's body, a long-stepped head screw is required. This screw is constituted by two cylindrical volumes superimposed, the upper one being wider, and lower by a cylindrical narrower stem with external threads. This screw also has a polyhedral cavity in its head that serves to manipulate it with the appropriate instrument.

This drill guide allows us to drill narrower holes in the bone than the inclined holes' width of in the tripod's body without touching it. We can also have a set of drill guides that allows to work bone depending of its density and hardness found when drilling. Once the bone has been drilled, the drill guide and fixing guide are removed so that the legs of the tripod can be inserted.

The legs of the tripod have a cylindrical head with a polyhedral cavity in its centre and below, a trunk also cylindrical. We will use the corresponding tripod leg depending on the chosen tripod's body type, thus, for a tripod's body with double width inclined perforations, we will insert some legs with wider heads than their trunks and, for a tripod body with a single space's inclined perforations and ditches near its entrances, we will push some tripod legs with heads of the same width as their trunks, heads that in this case, carry coupling rails parallel to their central axis and polymorphous perforations. These perforations are perpendicular to the central axis of these legs and in contact with their polyhedral cavity and serve to be able to pull these legs if necessary, with the suitable instrument. Three identical legs must be inserted to configure a perfect tripod that distributes loads properly. With the legs already placed on the bone, we can place a prosthesis on the tripod, where appropriate, thus serving this tripod as a short dental implant.

### BRIEF DESCRIPTION OF THE DRAWINGS

The description is supplemented, for an easy understanding of this description, with a set of drawings in which, with an illustrative and non-limiting character, the elements of the invention used during the fixing phase of the tripod body are represented as follows:

FIG. 1 represents a perspective side view of the two separate elements necessary for the use of the invention in its first phase of use. Here we see above the fixing guide (1) and below the tripod's body (2A) in its first variant. Above the guide (1) we see the screw (3) with simple cylindrical head (4) and stem (5) with external screw threads. This arrangement allows us to see the chalice (6) of the fixation guide (1) with its three guide rails (7A, 7B and 7C) and the polyhedral cavity (8) of the fixation guide (1). Below we see the cylindrical wall (9A) and the cylindrical floor (10A) of the tripod's body (2A). We also see the polyhedral foot (11) of the fixation guide (1) and the polyhedral cavity (12A) of the tripod's body (2A).

FIG. 2 represents a side view of a sagittal cut in perspective of the two separate elements necessary for the use of the invention in its first phase. Here we see above the fixing guide (1) and below the tripod's body (2A) in the first of its two variants. Above the fixing guide (1) we see the screw (3) with a simple cylindrical head (4) and stem (5) with external screw threads. This arrangement allows us to see, the calyx (6) of the fixation guide (1), a sagittal section of the orientation rail (7A), the orientation rail (7B) and the polyhedral cavity (8) of the fixation guide (1). Below we see the cylindrical wall (9A) and the floor (10A) of the tripod's body (2A) in one of its variants. We also see the polyhedral foot (11) of the fixation guide (1) and the polyhedral cavity (12A) of the tripod's body (2A) in one of its variants. Here we can see more detailed four holes of the fixing guide (1): a hole inclined (13A) in sagittal section, the entrance of another inclined hole (13B), its fourth hole (14) with its annular edge (15) and its fifth polyhedral hole (16). We also see the polyhedral cavity (17) of the screw (3). We also see four perforations of the tripod's body (2A) in one of its variants: an inclined perforation (18AA) represented with a wide upper part (19A) and a narrow lower part (19B) with propelled threads both separated by an annular edge (20); the entrance of another inclined perforation (18BA), its fourth perforation (21A) with propeller threads and its fifth perforation (22A) with propeller threads.

FIG. 3 represents a side view of a sagittal cut in perspective of the second variant of the tripod's body or alternative body (2B), represented with an inclined perforation (18AB) of a single space (23) with a trench (24) on the wall. We also see the polyhedral cavity (12B), the cylindrical wall (9B) and the floor (10B) of the alternative body (2B) of the tripod. We also see, his fourth perforation (21B) with propeller threads and his fifth perforation (22B) with propeller threads.

FIG. 4 represents a top view of the fixing guide (1) where we see the chalice (6) of the fixing guide (1) with its three guide rails (7A, 7B and 7C) and the polyhedral cavity (8) of the fixation guide (1). We see the entrances of the three inclined holes (13A, 13B and 13C) of the fixing guide (1), its fourth hole (14) with its annular edge (15) and its fifth polyhedral hole (16).

FIG. 5 represents a top view of the first variant of the body's tripod (2A), where we see its cylindrical wall (9A) and its polyhedral cavity (12A). We also see the entrances of the three inclined perforations (18AA, 18BA and 18CA) of the body (2A) of the tripod in one of its variants, its fourth perforation (21A) and its fifth perforation (22A) cylindrical.

FIG. 6 represents a perspective side view of the drill guide (25) where we can see, the handle (26) of this guide (25) and the four entrances of its four holes: the three entrances of its three inclined holes (27A, 27B and 27C) and the entrance of its fourth hole (28). We can also see the projecting polyhedral pivot (29) of the lower base of this drill guide (25).

FIG. 7 represents a top view of the drill guide (25) where we see its handle (26) and the four entrances of its four holes: the three entrances of its three inclined holes (27A, 27B and 27C) and the entrance of its fourth hole (28) with its annular edge (30).

FIG. 8 represents a perspective side view of the long and stepped head (32) screw (31) as well as its stem (33). We also see the polyhedral cavity (34) of the screw (31).

FIG. 9 represents a side view of a sagittal cut in perspective of the three separate elements necessary for the use of the invention in its second phase of use, the fixing guide (1), the first variant of the body (2A) of the tripod and the drill guide (25) together with the corresponding screw (31). Here we see above the drill guide (25) its handle (26), the inclined hole (27A), the entrance of the inclined hole (27B), its fourth hole (28) with its annular edge (30). We can also see the projecting polyhedral pivot (29) of the lower base of this drill guide (25). Above the drill guide (25) we see the long and stepped head (32) screw (31) with its polyhedral cavity (34) as well as its stem (33).

FIG. 10 shows a perspective side view of a possible leg (35) of the tripod here with a cylindrical head (36) having a polyhedral cavity (37) at its centre and a smooth or rough trunk (38). We see the coupling rails (39) of the head (36), here two, and also a polymorphous piercing (40) of the head (36).

FIG. 11 shows a side perspective view of another possible leg (41) of the tripod here with a cylindrical head (42) having a polyhedral cavity (43) at its centre and a narrower trunk (44) with external helical threads.

FIG. 12 shows the two stages precision guide (45) for fixing a tripod with short dental implant function with its two elements, the guide (1) for fixing the body (2A) of the tripod in one of its variants and the drill guide (25).

### PREFERENTIAL REALIZATION OF THE INVENTION

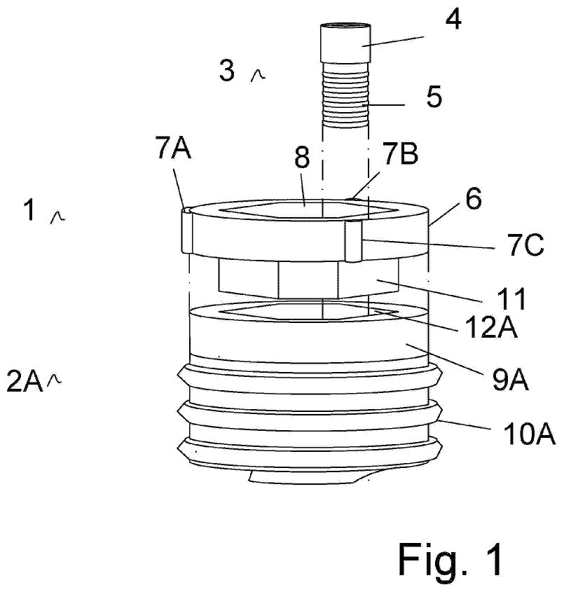

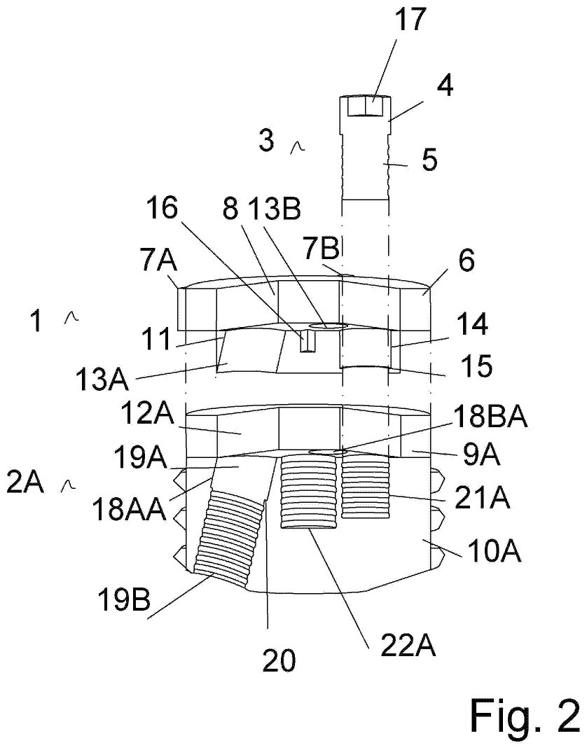

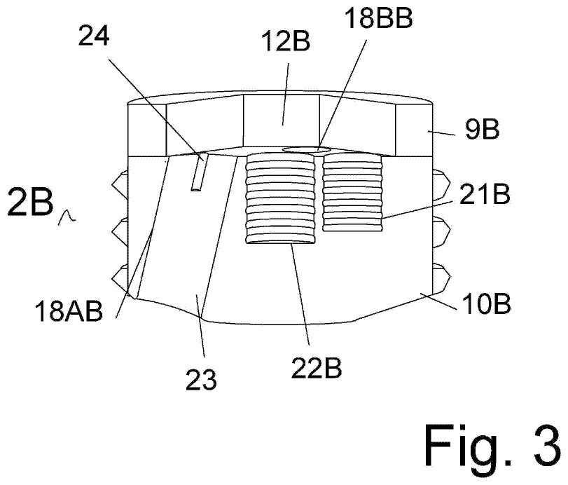

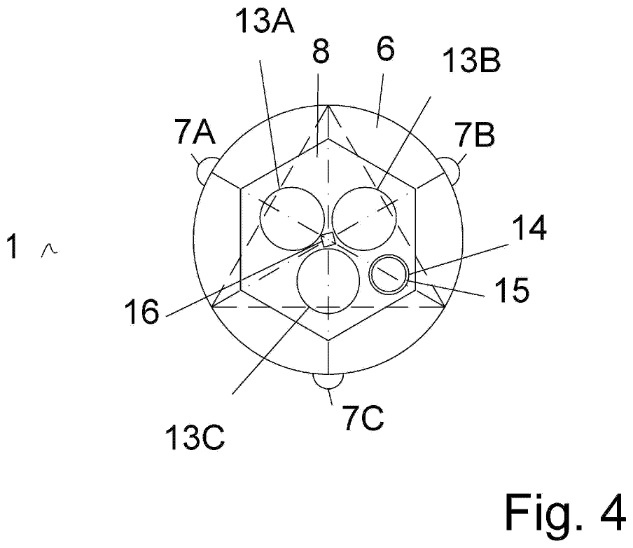

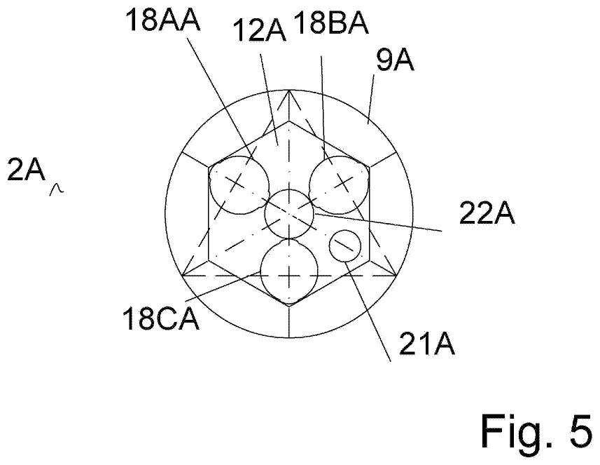

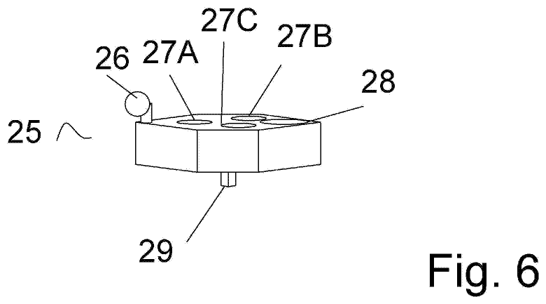

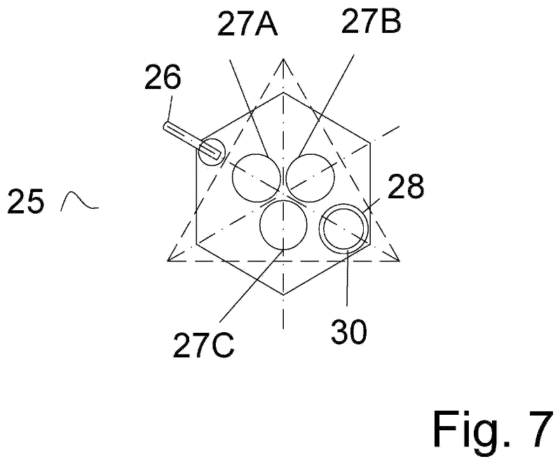

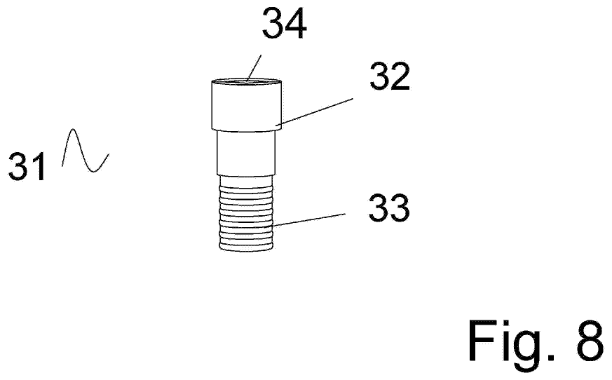

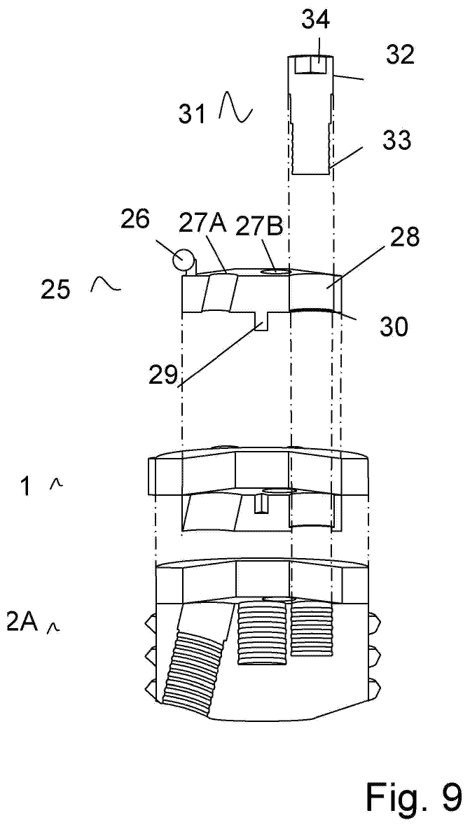

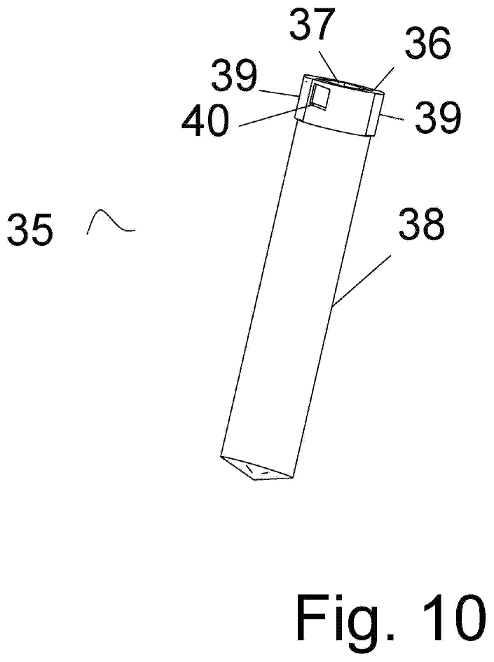

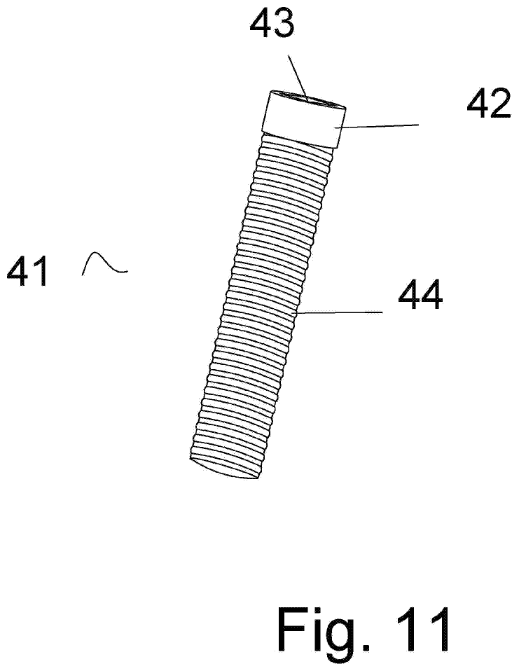

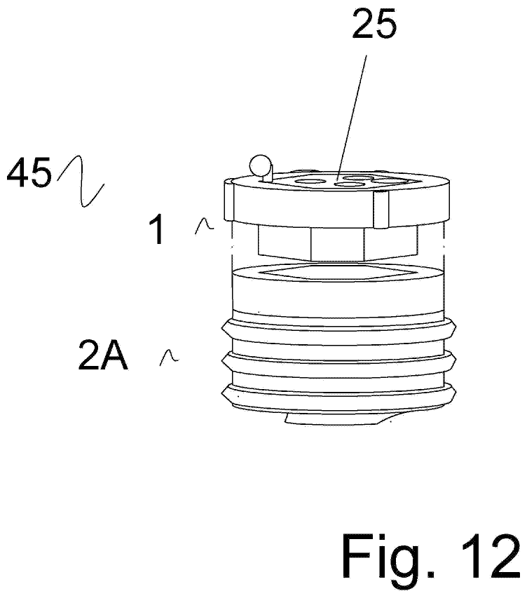

As already indicated, and as can be seen in FIG. 1, in its first phase of use, the two stages precision guide (45) to fix a tripod with short dental implant function, in a low jaw bone, object of the invention, in its preferred embodiment, comprises above the fixing guide (1) and below the body (2A) of the tripod, in the first of its two variants, joined by the simple cylindrical head (4) screw (3) and its stem (5) with external screw threads. More detailed, we see the chalice (6) of the fixation guide (1) with its three guide rails (7A, 7B and 7C) and the polyhedral cavity (8) of the fixation guide (1). Below we see the cylindrical wall (9A) and the cylindrical floor (10A) of the tripod's body (2A) in one of its variants. We also see the polyhedral foot (11) of the fixation guide (1) and the polyhedral cavity (12A) of the tripod's body (2A) in the first of its variants. In FIG. 2 we see the same objects of FIG. 1 with a sagittal cut which allows to appreciate in more detail some holes of the fixation guide (1) and of the tripod's body (2A) in the first of its variants: an inclined hole (13A) of the fixation guide in sagittal section, the entrance of another inclined hole (13B), its fourth hole (14) with its annular edge (15) and its fifth polyhedral hole (16). We also see the polyhedral cavity (17) of the screw (3). We also see four perforations of the tripod's body (2A) in the first of its variants: an inclined perforation (18AA) represented with a wide upper part (19A) and a narrow lower part (19B) with helical threads separated by an annular edge (20); the entrance of another inclined hole (18BA), its fourth hole (21A) with propeller threads and its fifth hole (22A) with propeller threads. In FIG. 3 we see the second variant in a sagittal section of a tripod's body or alternative body (2B), with an inclined perforation of a single space (23) and a trench (24) in its wall. We also see the polyhedral cavity (12B), the cylindrical wall (9B) and the floor (10B) of the alternative tripod's body (2B). We also see, his fourth perforation (21B) with propeller threads and his fifth perforation (22B) with propeller threads. In FIG. 4 we see a top view of the fixation guide (1) that allows us to see the proportions of this guide as well as its calyx (6), its four orientation rails (7A, 7B and 7C), its polyhedral cavity (8), the entrances of its three inclined holes (13A, 13B and 13C), its fourth hole (14) with its annular edge (15) and its fifth polyhedral hole (16). In FIG. 5 we see a top view of the tripod's body (2A) in its first variant, which allows us to see the proportions of this as well as its cylindrical wall (9A), its polyhedral cavity (12A), the entrances of its three perforations inclined (18AA, 18BA and 18CA), its fourth perforation (21A) and its fifth perforation (22A). In FIG. 6 in its second phase of use, the precision two-stage guide (45) for fixing a tripod with short dental implant function, in a low-lying maxillary bone, object of the invention, in its preferred embodiment, comprises the drill guide (25) with its handle (26) and the four entrances of its four holes: the three entrances of its three inclined holes (27A, 27B and 27C) and the entrance of its fourth hole (28). It also shows the outgoing polyhedral pivot (29) of its lower base. In FIG. 7 we see a top view of the drill guide (25) where we see its handle (26) and the four entries of its four holes: the three entrances of its three inclined holes (27A, 27B and 27C) and the entrance of its fourth hole (28) with its annular edge (30). In FIG. 8 we see the long and stepped head (32) screw (31), its polyhedral cavity (34) and its stem (33). In FIG. 9 we see in more detail the three separate elements necessary for the second phase of use of the invention, the fixing guide (1), the tripod's body (2A) in its first variant, and the drill guide (25) together with the corresponding screw (31). Here we see above the drill guide (25) where we see its handle (26), the inclined hole (27A), the entrance of the inclined hole (27B), its fourth hole (28) with its annular edge (30). We can also see the projecting polyhedral pivot (29) of the lower base of this drill guide (25). Above the drill guide (25) we see the long and stepped head (32) screw (31) with its polyhedral cavity (34) as well as its stem (33). In FIG. 10 we see a possible leg (35) of the tripod here with a cylindrical head (36) having a polyhedral cavity (37) at its centre and a smooth or rough trunk (38). We see the coupling rails (39) of the head (36), here two, and also a polymorphous piercing (40) of the head (36). In FIG. 11 we see another possible leg (41) of the tripod here with a cylindrical head (42) that has a polyhedral perforation (43) at its centre and a trunk (44) with external screw threads. In FIG. 12 we see the two-stage precision guide (45) for fixing a tripod with dental and short implant function, with its two components the fixation guide (1) and the drill guide (25) fixed to the tripod's body (2A) in the first of its variants.

What is claimed is:

1. A two-stage precision guide for fixing a tripod with a short dental implant function in a low-lying maxillary bone, the two-stage precision guide comprising:
a fixation guide and a drill guide,
wherein the fixation guide is a solid cup-shaped block extending along a central axis without a shaft and including a polyhedral foot having a cylindrical chalice, located above and extending about an outer surface of the polyhedral foot, and the polyhedral foot defining a polyhedral cavity opening upwards,
the polyhedral foot of the fixation guide defining five holes whereby these three of the five holes, spaced from one another and extending through said polyhedral foot, being inclined and having a cylindrical shape, identical to one another, central axes of the three of the five holes cross with the central axis of the fixation guide at a same point located above the fixation guide;
each of the three of the five holes having an inlet including an inlet center located at an upper base of the polyhedral foot of the fixation guide whereby the inlet centers, connected to one another, forms an equilateral triangle;
each of the three of the five holes having an outlet including an outlet center located at a bottom base of the polyhedral foot of the fixation guide whereby the outlet centers, connected to one another, forms a larger sized equilateral triangle,
the polyhedral foot of the fixation guide defining a fourth hole, having a cylindrical shape, located between two of the three holes, spaced from the two of the three holes extending parallel to the central axis, wherein the fourth hole tapers inside by an annular edge perpendicular to the central axis,
the fourth hole having an entrance and an exit with the entrance located in the upper base of the polyhedral foot and the exit located at the bottom base of the polyhedral foot whereby the entrance is wider than the exit,
the polyhedral foot further defining a fifth hole, having a polyhedral shape, extending along the central axis, surrounded by and spaced from the three holes and the fourth hole,
wherein the polyhedral cavity is delimited from below by the upper base of the polyhedral foot,
a plurality of three rails, each located in one of three sagittal planes of the three of the holes, and adhered to an outside wall of the chalice extending parallel to the central axis,
wherein the drill guide is a polyhedral slab having an exact same polyhedral shape as the polyhedral cavity of the fixation guide for placement in the polyhedral cavity;
the drill guide having an upper base and a bottom base with the bottom base including a polyhedral pivot projecting downwardly from a center of the bottom base whereby the polyhedral pivot has a shape that corresponds to the fifth hole of the fixing guide;
a handle, having a polymorphous shape and located at a corner of the upper base, protruding outwardly from the upper base;
the drill guide defining three holes, spaced from one another and being inclined, with each of the holes having a cylindrical shape and identical to one another, extending through the drill guide wherein central axes of each of the inclined holes intersect with a central axis of the drill guide at a same point located above the drill guide,
each of the three holes of the drill guide having an inlet including an inlet center located in the upper base of the drill guide whereby the inlet centers, connected to one another, forms an equilateral triangles,
each of the three holes of the drill guide having outlet including an outlet center located located in the lower base of the drill guide whereby the outlet centers, connected to one another, forms a larger sized equilateral triangle,
wherein the three holes of the drill guide have a width that is less than the width of the three of the five holes of the fixation guide and the outlets of the three holes of the drill guide are in connection with the inlets of the three of the fixation guide;
the drill guide defining a fourth hole, located between two of the three holes of the drill guide and having cylindrical shape, extending parallel to the central axis of the drill guide wherein the fourth hole of the drill guide tapers inside by an annular edge perpendicular to the central axis thereby reducing diameter of the fourth hole from an entrance of the fourth hole located on the upper base to an exit of the fourth hole located on the bottom base;
wherein a width of the outlet of the fourth hole of the drill guide is the same as a width of the entrance of the fourth hole of the fixation guide.

2. The two-stage precision guide according to claim 1, wherein the tripod comprises a central cylindrical body, having a shape of a wide-floor glass;
the central cylindrical body defining a polyhedral cavity opening upwards and surrounded by a cylindrical wall, having a smooth outer surface, the central cylindrical body including a floor having a rough surface and an external helical threads;
the floor of the central cylindrical body defining a plurality of five perforations located at the floor of the polyhedral cavity and extending from an upper base of the floor with three of the five perforations, being inclined and spaced from one another, each having a cylindrical shape, identical to one another, wherein central axes of the perforations intersect with a central axis of the central cylindrical body at a same point located above the central cylindrical body;
each of the three of the five perforations having an entry center, located at a bottom of the polyhedral cavity whereby the entry centers, connected to one another, forms an equilateral triangle;
each of the three of the five perforation having an outlet center located on the sides or at a lower base of the floor, whereby the outlet centers, connected to one another, forms a larger sized equilateral triangle,
each of the three of the five perforations being composed of two cylindrical spaces including an upper space and a lower space with the upper space being separated from the lower space by an annular edge, located perpendicular to a central axis of the perforation;
wherein the upper space is wider than the lower space and the lower space includes propeller threads located in the lower space;
the floor defining a fourth perforation, having a cylindrical shape, located between two of the three perforations of the cylindrical body and spaced from the two of the three perforations, extending parallel to the central axis of the central cylindrical body which does not pass through the floor;
the fourth perforation of the central cylindrical body being enclosed by a wall containing internal helical threads;
the floor defining a fifth perforation, having a cylindrical shape, located at a center of the floor, surround by and spaced from the three perforations and the fourth perforation, wherein the fifth perforation does not extend through the floor and that the fifth perforation being surrounded by a wall including internal helix threads.

3. The two-stage precision guide according to claim 1, wherein the tripod comprises a central cylindrical body having a shape of a wide-floor glass;
the central cylindrical body containing a polyhedral cavity, opening upwards, and surrounded by a cylindrical wall, having a smooth outer surface, the central cylindrical body including a floor having a rough surface and an external helical thread;
the floor of the central cylindrical body defining a plurality of five perforations located in the polyhedral cavity and extending from an upper base of the floor of the central cylindrical body with three of the five perforations, being inclined and spaced from one another, each having a cylindrical shape, identical to one another, wherein central axes of the perforations intersect with a central axis of the central cylindrical body at a same point located above the central cylindrical body;
each of the three of the five perforations having an entry center located at a bottom of the polyhedral cavity of the central cylindrical body whereby the entry centers, connected to one another, forms an equilateral triangle;
each of the three of the five perforations having an outlet center, located on the sides or at an lower base of the floor of the central cylindrical body whereby the outlet centers, connected to one another, forms a larger sized equilateral;
each of the three of the five perforations being composed of a single cylindrical space with smooth walls containing ditches that start from entrances of the cylindrical space, spaced from one another parallel to central axes of each of the five perforations wherein the ditches do not entirely cover the perforations.

4. A method of using the two-stage guide (45) as described in claim 2, the method includes the following steps:
joining the fixation guide and the central cylindrical body of the tripod with the polyhedral foot of the fixation guide being rested in the polyhedral cavity of the central cylindrical body and the chalice resting on a top surface of the central cylindrical body of the tripod;
fixing the fixation guide to the central cylindrical body of the tripod by inserting a screw into the fourth hole of the fixation guide and the fourth perforation of the central cylindrical body of the tripod wherein the screw has a head portion and a stem portion with the head portion defining a polyhedral cavity and the stem portion, being narrower than the head portion, including external helical threads;
introducing the central cylindrical body of the tripod the bone and positioning the central cylindrical body by arranging the three rails in the chalice of the fixation guide;
removing the screw;
fixing the drill guide, the fixation guide, and the central cylindrical body of the tripod by inserting a screw into the fourth hole of the drill guide, the fourth hole of the fixation guide, and the fourth perforation of the central cylindrical body of the tripod wherein the screw has a long and stepped head defining a polyhedral cavity disposed therein and a cylindrical narrower stem with external propeller threads;
perforating the bone through the three holes of the drill guide;
removing the long and stepped head of the screw;
removing the two-stage guide;
introducing three legs of the tripod wherein each of the three legs has a cylindrical head defining a polyhedral cavity located at a center of the cylindrical head and cylindrical stem with the cylindrical stem being narrower than the cylindrical head.

5. A method of using the two-stage guide (45) as described in claim 3, the method includes the following steps:
joining the fixation guide and the central cylindrical body of the with the polyhedral foot of the fixation guide being rested in the polyhedral cavity of the central cylindrical body and the chalice resting on a top surface of the central cylindrical body of the tripod;
fixing the fixation guide to the central cylindrical body of the tripod by inserting a screw into the fourth hole of the fixation guide and the fourth perforation of the central cylindrical body of the tripod wherein the screw has a head portion and a stem portion with the head portion defining a polyhedral cavity and the stem portion, being narrower than the head portion, including external helical threads;
introducing the central cylindrical body of the tripod into the bone and position the central cylindrical body by arranging the three rails in the chalice of the fixation guide;
removing the screw;
fixing the drill guide, the fixation guide, and the central cylindrical body of the tripod by inserting a screw into the fourth hole of the drill guide, the fourth hole of the fixation guide, and the fourth perforation of the central cylindrical body of the tripod wherein the screw has a long and stepped head defining polyhedral cavity disposed therein and a cylindrical narrower stem with external propeller threads;
perforating the bone through the three holes of the drill guide;
removing the long and stepped head of the screw;
removing the two-stages guide;
introducing three legs of the tripod wherein each of the three legs includes a head and a trunk with the head having a same width as the trunks and the head contains rails parallel to a central axis of the legs and defining at least one polymorphous perforations perpendicular to the central axis of the legs and in communication with a polyhedral perforation.
